(19) United States
(12) Patent Application Publication
(10) Pub. No.: US 2024/0016430 A1
(43) Pub. Date: Jan. 18, 2024

(54) MEASURING AND IMPROVING ATTENTION

(71) Applicants: Universitat De Barcelona, Barcelona (ES); Institució Catalana De Recerca I Estudis Avançats, Barcelona (ES)

(72) Inventor: Hendrik Anne SUPÈR, Barcelona (ES)

(73) Assignees: Universitat De Barcelona, Barcelona (ES); Institució Catalana De Recerca I Estudis Avançats, Barcelona (ES)

(21) Appl. No.: 18/037,191

(22) PCT Filed: Nov. 16, 2021
(86) PCT No.: PCT/EP2021/081811

§ 371 Date: May 16, 2023

(30) Foreign Application Priority Data
Nov. 17, 2020 (EP) 20382992.4

(51) Int. Cl. A61B 5/16

(52) U.S. Cl. CPC A61B 5/168; A61B 5/163

(57) ABSTRACT

Methods are provided of measuring and improving attention of a person with first and second eyes. These methods include: displaying one or more stimuli aimed at attracting attention of the person; obtaining, through an eye tracker, positions of the first and second eyes over time; calculating, from the obtained positions of the first and second eyes, a gaze direction evolution for each of the eyes and corresponding angular velocity evolution for each of the gaze directions; determining a motion synchrony evolution of the first and second eyes by comparing the calculated angular velocity evolution of the first eye with the calculated angular velocity evolution of the second eye; and measuring the attention of the person depending on the determined motion synchrony evolution. Computer programs, systems and computer systems that are suitable to perform such methods of measuring and improving attention are also provided.

### TECHNICAL FIELD

This disclosure relates to methods of measuring and improving attention of a person with first and second eyes, as well as to computer programs, systems and computer systems that are suitable to perform such methods of measuring and improving attention.

### BACKGROUND

Methods for measuring attention and/or other more or less related cognitive behaviours are known. Most of them try to identify and measure many different eye behaviours (e.g. saccades, blinks, eyelid, gaze fixation, pupil dilation, vergence, etc.) to obtain conclusions about attention. A drawback of these types of methods may be that they usually include collecting big amounts of data and performing heavy calculations on said collected data, which may cause some inefficiency and, thus, may require powerful and expensive computing resources.

Another inconvenience may be that these methods do not produce clean indicators of attention, since they take into account many different eye behaviours, which may produce measurements mixing attention with other cognitive processes different from attention (such as e.g. perception, memory, experience, etc.). This overload in terms of mixed phenomena in the measurements may thus result in indicators of attention some way distorted by noise caused by phenomena different from attention.

A further drawback may be that these methods may take too long to obtain more or less reliable conclusions, since they usually measure states (e.g. attention related states) considering long time scales, which may be of several minutes. These long-time scales may be necessary for these methods as a consequence of that they consider different eye behaviours, some of which may introduce some disparities in the collected data. It seems to make sense that collecting very big amounts of data and performing complex calculations on said data may be aimed at attenuating/compensating in some way such disparities.

An aspect of the present disclosure is to provide new systems, methods and computer programs aimed at improving current manners of measuring and improving attention of a person.

### SUMMARY

In an aspect, a method of measuring and improving attention of a person with first and second eyes is provided. The method includes displaying one or more stimuli aimed at attracting attention of the person, and obtaining, through an eye tracker, positions of the first and second eyes over time. The method further includes calculating, from the obtained positions of the first and second eyes, a gaze direction evolution for each of the first and second eyes and corresponding angular velocity evolution for each of the gaze directions. The method still further includes determining a motion synchrony evolution of the first and second eyes by comparing the calculated angular velocity evolution of the first eye with the calculated angular velocity evolution of the second eye. The method yet further includes measuring the attention of the person depending on the determined motion synchrony evolution.

The method of measuring attention is thus based on an innovative use or application of measuring synchrony. Measuring synchrony is based on comparing the angular velocities of eye movement direction, which cannot be derived from e.g. the calculation of eye vergence or saccade direction. For instance, increase of synchrony of the eyes can concur with an increase or decrease of vergence.

Experiments related to methods of measuring attention according to the present disclosure hereof have allowed for finding a clear relation between eye synchrony and attention. It is here concluded that the level of synchrony is modulated as a function of the ability to capture attention and mental or cognitive processing of stimuli. Synchrony appears to be lower and or thin peak after visual stimulation, and this enhancement appears to correlate with bottom-up and top-down attention. The start of the modulation in eye synchrony appears to be locked to the onset of the stimulus, while the size of the synchrony appears to depend on the mental or attentional load that the stimulus receives or attracts.

The methods hereof, based on measuring and promoting eye synchrony, may also be very useful to improve any type of cognitive function(s) related to or requiring or including attention, such as e.g. memory, perception, decision making, etc.

In some examples, determining the motion synchrony evolution may include determining a motion synchrony fluctuation over time, and the attention of the person may be measured depending on the determined motion synchrony fluctuation over time. The higher the motion synchrony fluctuation is, the higher the measured attention of the person may be determined. The lower the motion synchrony fluctuation is, the lower the measured attention of the person may be determined.

In some implementations, determining the motion synchrony evolution may include determining a modulation of the motion synchrony over time, and the motion synchrony fluctuation may be determined depending on one or more features of the motion synchrony modulation over time. The one or more features of the motion synchrony modulation may include at least one of amplitude, width of correlation peak, onset latency and duration.

According to examples, the method may further include determining, from the obtained positions of the first and second eyes, one or more eye fixations within at least some of which some restriction(s) may be defined. In an example, calculating the gaze direction evolutions and corresponding angular velocity evolutions may be performed exclusively within all or part of the determined one or more eye fixations. In another example, determining the motion synchrony evolution may be performed exclusively within all or part of the determined one or more eye fixations. In a further example, measuring the attention of the person may be performed exclusively within all or part of the determined one or more eye fixations.

In implementations of the method, displaying the one or more stimuli may include displaying first and second stimulus, and measuring the attention of the person may include measuring attention within first fixation to the first stimulus and attention within second fixation to the second stimulus. In particular, measuring the attention of the person may include comparing the attention within first fixation and the attention within second fixation to determine which of the first and second stimulus has been most attended and/or to which extent one of the first and second stimulus has been more attended than the other.

As described in other parts of the present disclosure, performed experiments revealed that the level of synchrony appears to modulate as a function of the ability to capture attention and mental or cognitive processing of stimuli. This modulation was found to be more distinguishable and, therefore, better detectable and processable within eye fixation(s). Hence, restricting the measuring of synchrony (and, finally, attention) to within eye fixation(s) permits obtaining more reliable indicators or measurements of attention.

In some examples, a videogame may be provided including any one of the disclosed methods of measuring and improving attention, with one of the one or more stimuli of the method of measuring and improving attention that may correspond to an eye-controllable item depending on the measured attention to said eye-controllable item.

In a further aspect, a computer program is provided including program instructions for causing a computing system to perform any one of the disclosed methods of measuring and improving attention and videogame. The computer program may be embodied on a storage medium and/or carried on a carrier signal.

In a furthermore aspect, use of a videogame is provided for improving attention of a person, said videogame being an eye-controlled video game or including an eye-controlled item or aspect in the video game. Any fully or partially eye-controlled video game may be used to improve attention of a corresponding player or user or person.

In a still further aspect, a system is provided for measuring attention of a person with first and second eyes. The system includes a display sub-system, an eye-positions sub-system, an angular-speed sub-system, a synchrony sub-system, and a measuring sub-system. The display sub-system is configured to display one or more stimuli aimed at attracting attention of the person. The eye-positions sub-system is configured to obtain, through an eye tracker, positions of the first and second eyes over time. The angular-speed sub-system is configured to calculate, from the obtained positions of the first and second eyes, a gaze direction evolution for each of the first and second eyes and corresponding angular velocity evolution for each of the gaze directions. The synchrony sub-system is configured to determine a motion synchrony evolution of the first and second eyes by comparing the calculated angular velocity evolution of the first eye with the calculated angular velocity evolution of the second eye. The measuring sub-system is configured to measure the attention of the person depending on the determined motion synchrony evolution.

### BRIEF DESCRIPTION OF DRAWINGS

Non-limiting examples of the present disclosure will be described in the following, with reference to the appended drawings, in which:

FIGS. 1a and 1b show schematic representations of a first and a second system for measuring attention, according to examples.

FIG. 2 is a flow chart schematically illustrating methods of measuring and improving attention of a person, according to examples.

FIG. 3 is a schematic illustration of principles usable to calculate angular velocity evolutions of first and second eyes' gaze, according to examples.

### DETAILED DESCRIPTION

In these figures the same reference signs have been used to designate same or similar elements.

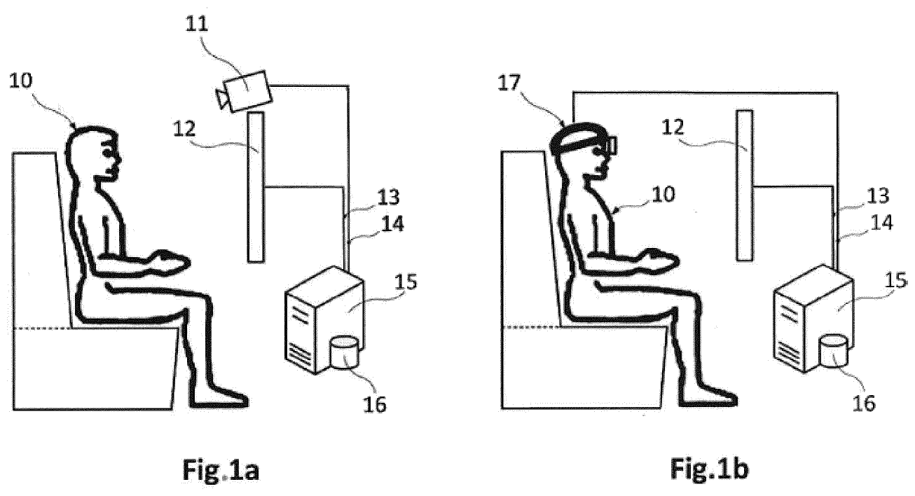

FIG. 1a illustrates a system for measuring attention of a person 10 with first and second eyes. This system may include a display sub-system, an eye-positions sub-system, an angular-speed sub-system, a synchrony sub-system, and a measuring sub-system. The display sub-system may include a screen 12 to display one or more stimuli aimed at attracting attention of the person 10. The eye-positions sub-system may include an eye position tracker, such as e.g. a suitable camera 11, to obtain positions of the first and second eyes over time. In other applications and/or configurations, the eye position tracker may be implemented by/in or based on a webcam and/or selfie cam of e.g. a smartphone or similar device.

A computing system 15 may be configured to execute a computer program configured to perform any one of the methods of measuring and improving attention disclosed herein. The computer 15 may include a repository, such as a conventional hard disk 16, to store and retrieve data produced and/or required by said computer program. Such a computer program may therefore include any piece of software needed to perform corresponding method of measuring and improving attention.

One piece of software or sub-system in the computer program may be configured to generate the one or more stimuli to send them to the screen 12. This one piece of software and the screen 12 may thus form the display sub-system of the system for measuring attention. Another piece of software or sub-system in the computer program may be configured to obtain, through the camera 11, the positions of the first and second eyes over time. The camera 11 may be positioned in such a way that positions of the first and second eyes of the person 10 may be properly captured. This other piece of software and the camera 11 may thus form the eye-positions sub-system of the system for measuring attention.

Both the camera 11 and the screen 12 may be suitably connected 13, 14 with the computer 15, such that the computer 15 may interchange suitable signals with the screen 12 mainly to present the corresponding visual stimulus, and may interchange suitable signals with the camera 11 mainly to obtain positions of the eyes. Said connections 13, 14 may be wired and/or wireless connections.

A further piece of software or sub-system in the computer program may be configured to calculate, from the positions of the first and second eyes (from the eye-positions sub-system), a gaze direction evolution for each of the first and second eyes and corresponding angular velocity evolution for each of the gaze directions. This further piece of software may therefore correspond to the angular-speed sub-system of the system for measuring attention.

A still further piece of software or sub-system in the computer program may be configured to determine a motion synchrony evolution of the first and second eyes by comparing the calculated angular velocity evolutions of the first and second eyes (from the angular-speed sub-system) with each other. This still further piece of software may accordingly correspond to the synchrony sub-system of the system for measuring attention.

A yet further piece of software or sub-system in the computer program may be configured to measure the attention of the person depending on the determined motion synchrony evolution (from the synchrony sub-system). This yet further piece of software may hence correspond to the measuring sub-system of the system for measuring attention.

Systems for measuring attention according to FIG. 1a may further include proper apparatuses, methods and/or systems to hold the head of the person 10 in a substantially fixed position. These fixation apparatuses, methods and/or systems are not shown in the figure. Alternatively, the system may not include such apparatuses, methods and/or systems, in which case the camera 11 may include a suitable software (or computer program) to evaluate the movements of the head of the person 10. Since movements of the head may distort in some way the captured movements of the eyes, this software may take into account the movements of the head to attenuate said potential distorts. According to this, the camera 11 may thus be able to send “clean” positions of the eyes to the computer 15. Alternatively, the camera 11 may not include such software, in which case an equivalent software may be included in the computer 15. In this case, the camera 11 may only send to the computer 15 signals representing captured images of the head (including the eyes) and the computer 15 may attenuate, through said equivalent software, potential distortions due to movements of the head.

FIG. 1b illustrates another system for measuring attention of the person 10 similar to the system of FIG. 1a. A difference between them is that the system of FIG. 1b includes an eye tracker 17 which is different from the eye tracker 11 of FIG. 1a. In this case, a software for compensating movements of the head may not be necessary because the head-mounted eye tracker 17 moves jointly with the head.

Alternatively to camera 11 and to head-mounted tracker 17, examples of the system may include a device suitable for performing electro oculography (EOG). And, alternatively, examples of the system may include scleral coils.

In general, eye trackers necessarily measure the motion (e.g. rotation) of the eyes with respect to the measuring system. If the measuring system is head mounted, such as the device 17 of FIG. 1b, eye-in-head angles may be measured. If the measuring system is table mounted, such as the camera 11 of FIG. 1a, then gaze angles may be measured.

One of the most widely used current designs may be video-based eye trackers. A camera focuses on one or both eyes and records their movement as the viewer looks at some kind of stimulus. Most modern eye-trackers may use contrast to locate the centre of the pupil and use infrared and near-infrared non-collimated light to create a corneal reflection. The vector between these two features may be used to compute gaze intersection with a surface after a simple calibration for an individual.

Light, typically infrared, may be reflected from the eye and sensed by a video camera or some other specially designed optical sensor. The information may then be analysed to extract eye motion/rotation from changes in reflections. Video based eye trackers may typically use the corneal reflection and the centre of the pupil as features to be tracked over time. A more sensitive type of eye tracker may use reflections from the front of the cornea and the back of the lens as features to be tracked. A still more sensitive tracking method may include imaging features from inside the eye, such as the retinal blood vessels, and follow these features as the eye moves/rotates.

The systems of FIGS. 1a and 1b may hence be suitable for performing any one of the methods of measuring and improving attention according to present disclosure.

Angular velocity evolution of each eye and corresponding inter-eye motion synchrony evolution may be calculated from the obtained positions of the first and second eyes by applying any suitable known algorithm aimed at that. Said algorithms, which may be mainly based on trigonometric calculations, are well known, so no particular details about them will be provided herein.

The motion synchrony evolution may include e.g. a motion synchrony fluctuation over time and, in said case, the attention of the person may be measured depending on said motion synchrony fluctuation determined (by e.g. the synchrony sub-system) over time. Then, the attention may be measured (by e.g. the measuring sub-system) directly proportional to the motion synchrony fluctuation. The higher the motion synchrony fluctuation is determined, the higher the attention may be measured, and vice versa, i.e. the lower the motion synchrony fluctuation is determined, the lower the attention may be measured.

The motion synchrony evolution may be determined (by e.g. the synchrony sub-system) by determining a motion synchrony modulation over time. In particular, the motion synchrony evolution may be determined depending on one or more features of such a motion synchrony modulation. Said one or more features of the motion synchrony modulation may include e.g. an amplitude, and/or a correlation peak width, and/or an onset latency, and/or a duration.

Methods of measuring and improving attention according to the present disclosure may further include determining one or more eye fixations depending on the positions of first and second eyes (obtained by e.g. the angular-speed sub-system). Then, the measuring of the attention may be restricted to at least some of said eye fixations. This restriction to eye fixation(s) may be implemented by e.g. calculating gaze direction evolutions and angular velocity evolutions, and/or determining motion synchrony evolution, and/or measuring the attention itself exclusively within all or part of the determined eye fixation(s).

The one or more stimuli may include first and second stimuli to cause a first fixation to the first stimulus and second fixation to the second stimulus, such that attention may be measured within first fixation and within second fixation. Then, attention measured within first and second fixations may be compared with each other to determine which of the first and second stimuli has been most attended, and/or to which extent one of the first and second stimuli has been more attended than the other.

Videogames based on, or including, any of the proposed methods of measuring and improving attention may be also provided. In these videogames, one of the stimuli of the measuring method may correspond to an eye-controllable item, whose display may be controlled depending on the attention by user/person to said eye-controllable item as measured by the measuring method.

As used herein, the term “sub-system” may be understood to refer to software, firmware, hardware and/or various combinations thereof. It is noted that the sub-systems are exemplary. The sub-systems may be combined, integrated, separated, and/or duplicated to support various applications. Also, a function described herein as being performed by a particular sub-system may be performed by one or more other sub-systems and/or by one or more other devices instead of or in addition to the function performed by the described particular sub-system.

The sub-systems may be implemented across multiple devices, associated or linked to corresponding methods of measuring and improving attention proposed herein, and/or to other components that may be local or remote to one another. Additionally, the sub-systems may be moved from one device and added to another device, and/or may be included in both devices, associated to corresponding methods of measuring and improving attention proposed herein. Any software implementations may be tangibly embodied in one or more storage media, such as e.g. a memory device, a floppy disk, a compact disk (CD), a digital versatile disk (DVD), or other devices that may store computer code.

The methods of measuring and improving attention according to the present disclosure may be implemented by computing apparatuses, methods and/or systems or sub-systems, electronic apparatuses, methods and/or systems or sub-systems or a combination thereof. The computing apparatuses, methods and/or systems or sub-systems may be a set of instructions (e.g. a computer program) and then the methods of measuring and improving attention may include a memory and a processor, embodying said set of instructions stored in the memory and executable by the processor. These instructions may include functionality or functionalities to execute corresponding methods of measuring and improving attention such as e.g. the ones described with reference to the figures.

In case the methods of measuring and improving attention are implemented only by electronic apparatuses, methods and/or systems, a controller of the system may be, for example, a CPLD (Complex Programmable Logic Device), an FPGA (Field Programmable Gate Array) or an ASIC (Application-Specific Integrated Circuit).

In case the methods of measuring and improving attention are a combination of electronic and computing apparatuses, methods and/or systems, the computing apparatuses, methods and/or systems may be a set of instructions (e.g. a computer program) and the electronic apparatuses, methods and/or systems may be any electronic circuit capable of implementing corresponding method-steps of the methods of measuring and improving attention proposed herein, such as the ones described with reference to figures.

The computer program(s) may be embodied on a storage medium (for example, a CD-ROM, a DVD, a USB drive, a computer memory or a read-only memory) or carried on a carrier signal (for example, on an electrical or optical carrier signal).

The computer program(s) may be in the form of source code, object code, a code intermediate source and object code such as in partially compiled form, or in any other form suitable for use in implementing the methods of measuring and improving attention according to the present disclosure. The carrier may be any entity or device capable of carrying the computer program(s).

For example, the carrier may include a storage medium, such as a ROM, for example a CD ROM or a semiconductor ROM, or a magnetic recording medium, for example a hard disk. Further, the carrier may be a transmissible carrier such as an electrical or optical signal, which may be conveyed via electrical or optical cable or by radio or other apparatuses, methods and/or systems.

When the computer program(s) is/are embodied in a signal that may be conveyed directly by a cable or other device or apparatuses, methods and/or systems, the carrier may be constituted by such cable or other device or apparatuses, methods and/or systems. Alternatively, the carrier may be an integrated circuit in which the computer program(s) is/are embedded, the integrated circuit being adapted for performing, or for use in the performance of, the methods of measuring and improving attention proposed herein.

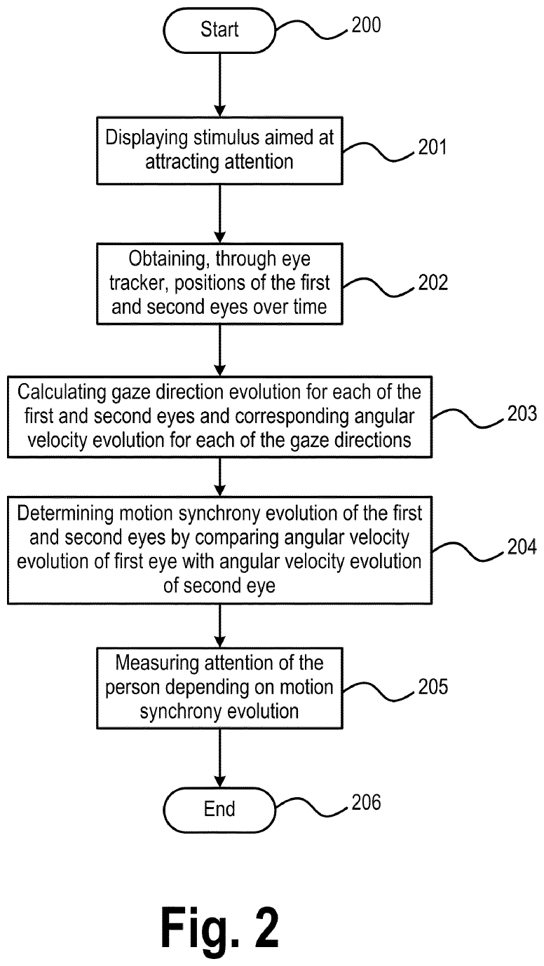

FIG. 2 is a flow chart schematically illustrating methods of measuring and improving attention of a person, according to examples. As generally shown in the figure, such “measuring” methods may be initiated (e.g. at method block 200) upon detection of a starting condition such as e.g. a user request for starting the method, initiation of system for measuring attention, etc. Since methods according to FIG. 2 are performable by systems according to FIGS. 1a and 1b, number references from said figure may be reused in following description of FIG. 2.

Measuring methods may further include (e.g. at method block 201) displaying one or more stimuli aimed at attracting attention of the person. Since this functionality implementable at e.g. method block 201 is performable by e.g. the display sub-system 12 of the system for measuring attention, functional details and considerations previously explained in this respect (with reference to previous FIGS. 1a and 1b) may thus be similarly attributed to method block 201.

Measuring methods may still further include (e.g. at method block 202) obtaining, through an eye tracker 11, 17, positions of the first and second eyes over time. Since this functionality implementable at e.g. method block 202 is performable by e.g. the eye-positions sub-system 11, 17 of the system for measuring attention, functional details and considerations previously explained in this respect (with reference to previous FIGS. 1a and 1b) may thus be similarly attributed to method block 202.

Measuring methods may yet further include (e.g. at method block 203) calculating, from positions of the first and second eyes (obtained at e.g. previous block 202), a gaze direction evolution for each of the first and second eyes and corresponding angular velocity evolution for each of the gaze directions. Since this functionality implementable at e.g. method block 203 is performable by e.g. the angular-speed sub-system (in e.g. computer 15) of the system for measuring attention, functional details and considerations previously explained in this respect (with reference to previous FIGS. 1a and 1b) may thus be similarly attributed to method block 203.

Measuring methods may furthermore include (e.g. at method block 204) determining a motion synchrony evolution of the first and second eyes by comparing the calculated angular velocity evolution of the first and second eyes (from e.g. previous block 203) with each other. Since this functionality implementable at e.g. method block 204 is performable by e.g. the synchrony sub-system (in e.g. computer 15) of the system for measuring attention, functional details and considerations previously explained in this respect (with reference to previous FIGS. 1a and 1b) may thus be similarly attributed to method block 204.

Measuring methods may furthermore include (e.g. at method block 205) measuring the attention of the person depending on the motion synchrony evolution determined at e.g. previous block 204. Since this functionality implementable at e.g. method block 205 is performable by e.g. the measuring sub-system (in e.g. computer 15) of the system for measuring attention, functional details and considerations previously explained in this respect (with reference to previous FIGS. 1a and 1b) may thus be similarly attributed to method block 205.

Measuring methods may still furthermore include (e.g. at method block 206) terminating execution of the method when e.g. an ending condition is satisfied. Ending condition satisfaction may be determined by detecting e.g. a user request for ending the method, or turning off of the system for measuring attention, etc.

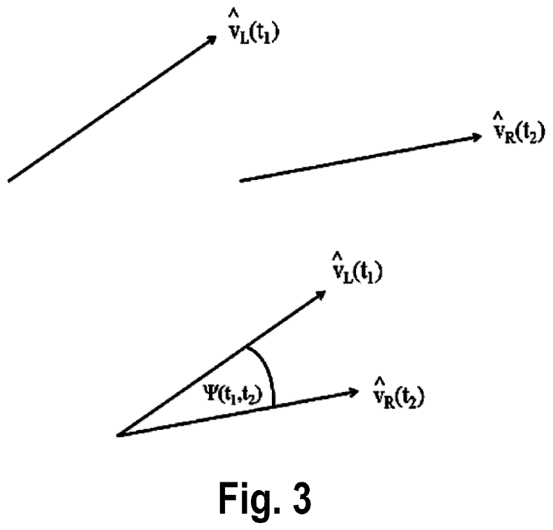

FIG. 3 is a schematic illustration of principles usable to calculate angular velocity evolutions of first and second eyes' gaze, according to examples. It is shown in the figure that two pairs of coordinates may form two trajectories in image plane. From two gaze direction vectors or trajectories (of first and second eyes' gaze), directions of motion may be determined in that plane at every time (e.g. continuously), and the angle between the two directions may be considered. Thus, alignment between the first and second eye motions may be described. In geometrical terms, the target angle may be the one formed by the tangent vectors to the two trajectories, i.e. an angle Ψ(t1, t2) formed by or between two velocity vectors \hat{V}_{L} (t₁)\hat{V}_{R}(t₂)(not by the gaze vectors themselves, but by velocity vectors originated by motion of gaze vectors). In a geometrical interpretation, symbols \hat{V}_{L} (t₁)\hat{V}_{R}(t₂) may correspond to velocities of left and right (or first and second) eye velocities on the image plane, possibly taken in general at different times (top part of FIG. 3), and symbol Ψ(t1, t2) may correspond to the angle formed by/between directions of said two vectors \hat{V}_{L} (t₁)\hat{V}_{R}(t₂) (bottom part of FIG. 3).

Experiments were performed by testing persons in a figure-ground paradigm. During the task, the eye synchrony was measured. Surprisingly, the size of the synchrony was not constant, but it appeared to be affected by visual stimulation and cognitive process. Synchrony of eye movements appeared to change when perceiving a visual stimulus (“hit”) but not when the stimulus was unnoticed (“miss”). When persons reported perception of a non-existing figure (“false alarm”), similar changes in synchrony as true hits were observed. Conversely, when nothing was perceived (“correct rejection”), eye movement synchrony resembled a “miss” case. Micro saccades appeared to promote eye velocity synchrony whereas eye vergence appeared to desynchronize eye motion. Micro-saccades, in contrast to target saccades, were detected within fixation whereas target saccades were identified from one fixation to another.

The proposed methods of measuring attention, which attribute an innovative role to eye synchrony, have the advantage that only one type of eye behaviour (synchrony) may be used to conclude about how attentive is a person. In some, these methods may be restricted to eye fixation(s) within which only measured eye synchrony may be taken into account as an indicator of attention. Therefore, necessary data and calculations are drastically reduced in comparison with prior methods. Moreover, according to performed experiments, eye synchrony may be considered a reliable indicator of attention isolated from other “disturbing” cognitive processes. In conclusion, methods of measuring and improving attention according to the present disclosure may provide reliable results in a more efficient and clean manner in very short time. Few seconds of collected eye synchrony data may suffice to evaluate attention.

Another advantage of these methods may be that, once positions of the eyes have been obtained during presentation of the one or more stimuli, presence of the person is no more required. Then, the obtained positions of the eyes over time may be used to perform calculations for measuring eye synchrony (only within eye fixation(s), in some examples). Thus, in this context, the terms “detecting”, “determining”, “calculating”, “measuring” may refer to perform corresponding calculations on collected data (positions of the eyes over time) to detect eye fixations, in some examples, and associated eye synchrony.

Methods of measuring attention according to present disclosure may be of application to different fields, such as web usability, advertising, sponsorship, package design, automotive engineering, etc. Examples of target stimuli may include websites, television programs, sporting events, films, commercials, magazines, newspapers, packages, shelf displays, consumer systems, software, etc. The resulting data may be statistically analysed and graphically rendered to provide evidence of specific visual patterns. By examining eye synchrony as an indicator of attention, effectiveness of a given medium or product may be determined.

When a person looks at an image, he/she scans the image by making fast saccadic eye movements to particular regions in the scene and fixates for a brief period during which the visual information is perceived. However, not all fixations are perceived consciously and/or influence behaviour, and some are better observed than others. Methods according to fixation-based approach as defined in other parts of the disclosure may permit separating this difference between eyes fixation and perception.

While looking at an image (advertisement, webpage), the regions where the person looked at may be retrieved and the eye synchrony (within eye fixation(s) in fixation-based approaches) may be calculated and measured. Regions of interest that show large fluctuations in eye synchrony (within or outside fixation(s) or both) may be indicative of strongly attended regions and of better perceived regions, whereas regions where eye synchrony shows less fluctuations (within or outside fixation(s) or both) may be indicative of less attended or perceived regions, despite these regions may have been fixated (or not). Fluctuations in synchrony may be calculated according to any known method(s) aimed at said or similar purpose, which may be based on one or more features of synchrony modulation. Such features may include, e.g., amplitude, width of correlation peak, onset latency, duration, etc.

Therefore, methods according to present disclosure may be very useful for e.g. designing advertisements. For example, such methods may be performed taking into account different profiles of people watching a product to be advertised. Then, data may be provided about parts of the product that e.g. have been more attended by people, in such a way that conclusions about which parts and how salient they may be showed to attract maximum attention may be obtained.

When performing repetitive tasks, it is easy to become bored and overlook important details of the activity in progress. Many activities are repetitive especially in assembly work or when visually monitoring products or images. Methods according to the present disclosure may also be very useful to know when a task becomes boring and, thus, it may lead to mistakes. During many repetitive tasks, eye position(s) may be recorded and then eye synchrony may be measured from recorded data. The peak of the correlation peak may be then calculated during the initial start of the activity. This value may be used as a base line level. Then, during performance of the task, eye synchrony may be monitored and compared with the initial synchrony modulation. Modulation in eye synchrony, i.e. changes in correlation peak width and/or height, may reflect shift(s) in attention. Higher frequency in peak changes may mean frequent shifts of attention. If a person becomes bored, attention is shifted less times, although the eyes still may move. If the frequency in modulation of peak width and/or height drops below a certain threshold, then this may be indicative of less attentive state of the person, who may thus need to take a break or to be replaced.

Browsing the Internet or digital media or similar, people may be subjected to many types of stimulus (e.g. advertisements). Many of them may however be never noticed by the user. Methods of measuring (and improving) attention according to the present disclosure may be used to improve visibility of such stimulus (e.g. advertisements). When browsing or reading digital, eye movements may be recorded e.g. via a suitable webcam or selfie cam (acting as an eye tracker) and eye synchrony may be calculated. When thin peak synchrony occurs, attention may be interpreted as increased. This measure may be used to time at the moment of the presentation or alteration of the stimulus that is being monitored. It is more likely that the user notices the appearing stimulus when peak in eye synchrony is thinner and lower. Thus, effectiveness of presented stimulus in digital content may be improved by monitoring eye synchrony as defined in methods according to the present disclosure.

As described in other parts of the disclosure, videogames may be provided including any one of the disclosed methods of measuring and improving attention. In these video games, one of the one or more stimuli of the method of measuring and improving attention may correspond to an eye-controllable item which is controlled depending on the measured attention to said eye-controllable item. The more attended by user/player said eye-controllable item is, the more it may behave in the game in line with user/player's gaze. That is, the higher user/player's attention to said item is detected, the higher the control by user/player may be on the item. For example, high attention to the item may imply that the item moves in the game strictly aligned with gaze motion, and vice versa, i.e. less attended item my imply that item moves in the game less aligned with gaze motion.

Additionally or alternatively to the gaze- or eye-controlled video games including methods of measuring attention according to the present disclosure, any known gaze/eye-controlled video game may be used to improve cognitive and/or attentional and/or awareness capabilities of a corresponding player, user, viewer or person.

In gaze/eye-controlled video games, position and/or motion of gaze may be used as input to pertinent device to control the game. Gaze may be determined by e.g. fixation(s) which may be defined as the duration of relatively stable gaze where no target saccades occur. In proposed uses of a videogame to improve attention, synchrony and/or vergence measures may be used during a single (or each or some) gaze fixation to control video gaming and thereby improving attentional and/or cognitive behaviour. Principles commented about synchrony in other aspects of the present disclosure may be similarly applied to these uses of eye-controlled video game for improving attentional and/or awareness and/or cognitive capabilities of a person or user or player.

Experiments have been carried out by inventors which have revealed that playing action video games may enhance visual attention skills. Children who played action games improved their reading speed and also scored better on tests of visual attention. It was also confirmed that video games may reduce impulsivity behaviour in children suffering from e.g. ADHD when the game is controlled with their eye gaze, and not when using a mouse or joystick or similar device as controller. Gaze controlled games may improve the control of gaze fixation and direction of voluntary target saccades in patients with e.g. neurodevelopmental disorder like ASD, Dyslexia, and ADHD. Theoretically, activation and training of the voluntary saccade circuit subsequently activates and trains the spatial attention circuit.

It was also found that gaze/eye-controlled games may activate or modulate certain brain areas known to be affected in and/or origin of cognitive disorders such as ADHD, MCI and Alzheimer. Playing gaze/eye-controlled games may therefore reduce symptoms or even treat these disorders by inducing certain type of neural activity (e.g. gamma activity) which is known to change or improve biochemical processes (e.g., reduction of amyloid plaque and activation of microglial cells).

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples, but should be determined only by a fair reading of the claims that follow.

1. A method of measuring and improving attention of a person with first and second eyes, the method comprising:
displaying one or more stimuli aimed at attracting attention of the person;
obtaining, through an eye tracker, positions of the first and second eyes over time;
calculating, from the obtained positions of the first and second eyes, a gaze direction evolution for each of the first and second eyes and corresponding angular velocity evolution for each of the gaze directions;
determining a motion synchrony evolution of the first and second eyes by comparing the calculated angular velocity evolution of the first eye with the calculated angular velocity evolution of the second eye; and
measuring the attention of the person depending on the determined motion synchrony evolution.

2. A method according to claim 1, the determining the motion synchrony evolution further including determining a motion synchrony fluctuation over time; and the measuring the attention of the person further including measuring the attention of the person depending on the determined motion synchrony fluctuation over time.

3. A method according to claim 2, further comprising the higher a motion synchrony fluctuation indicating a higher measured attention, and the lower the motion synchrony fluctuation indicating a lower measured attention of the person.

4. A method according to any claim 2, wherein the determining the motion synchrony evolution further including determining a modulation of the motion synchrony over time; and determining the motion synchrony fluctuation is depending on one or more features of the motion synchrony modulation over time.

5. A method according to claim 4, the one or more features of the motion synchrony modulation including at least one of amplitude, width of correlation peak, onset latency and duration.

6. A method according to claim 1, further comprising determining, from the obtained positions of the first and second eyes, one or more eye fixations.

7. A method according to claim 6, the calculating the gaze direction evolutions and corresponding angular velocity evolutions being is-performed exclusively within all or part of the determined one or more eye fixations.

8. A method according to claim 6, the determining the motion synchrony evolution being performed exclusively within all or part of the determined one or more eye fixations.

9. A method according to claim 6, the measuring the attention of the person being performed exclusively within all or part of the determined one or more eye fixations.

10. A method according to claim 6, the displaying the one or more stimuli further including displaying first and second stimuli; and the measuring the attention of the person further including measuring attention within first fixation to the first stimulus and attention within second fixation to the second stimulus.

11. A method according to claim 10, the measuring the attention of the person further including comparing the attention within first fixation and the attention within second fixation to determine which of the first and second stimuli has been most attended and/or to which extent one of the first and second stimuli has been more attended than the other.

12. A videogame comprising a method as defined in claim 1, wherein the one of the one or more stimuli of the method of measuring and improving attention corresponding to an eye-controllable item depending on the measured attention to the eye-controllable item.

13. A computer program product comprising program instructions for causing a computer to perform a method as defined in claim 1.

14. A computer program according to claim 13, embodied on a storage medium and/or carried on a carrier signal.

15. A system for measuring attention of a person with first and second eyes, the system comprising:
a display sub-system configured to display one or more stimuli aimed at attracting attention of the person;
an eye-positions sub-system configured to obtain, through an eye tracker, positions of the first and second eyes over time;
an angular-speed sub-system configured to calculate, from the obtained positions of the first and second eyes, a gaze direction evolution for each of the first and second eyes and corresponding angular velocity evolution for each of the gaze directions;
a synchrony sub-system configured to determine a motion synchrony evolution of the first and second eyes by comparing the calculated angular velocity evolution of the first eye with the calculated angular velocity evolution of the second eye; and
a measuring sub-system configured to measure the attention of the person depending on the determined motion synchrony evolution.
